(19) United States
(12) Patent Application Publication
(10) Pub. No.: US 2002/0173447 A1
(43) Pub. Date: Nov. 21, 2002

(54) Cytostatic effects of fatty acid synthase inhibition

(76) Inventors: Ellen Sarah Pizer, Clarksville, MD (US); Francis Paul Kuhajda, Lutherville, MD (US); Craig A. Townsend, Baltimore, MD (US)

Correspondence Address: BROBECK, PHLEGER & HARRISON, LLP, ATTN: INTELLECTUAL PROPERTY DEPARTMENT, 1333 H STREET, N.W. SUITE 800, WASHINGTON, DC, 20005, US

(21) Appl. No.: 10/075,344

(22) Filed: Feb. 15, 2002

Related U.S. Application Data
(60) Provisional application No. 60/268,680, filed on Feb. 15, 2001.

(51) Int. Cl. A61K 31/00

(52) U.S. Cl. 514/1

(57) ABSTRACT

This invention provides a method for treating an individual having a tumor by administering to the individual an inhibitor of fatty acid synthase (FAS) in an amount sufficient to retard growth of cells in the tumor. Preferably, the method of this invention is applied to an individual having a tumor comprising cells which do not overexpress FAS or a tumor comprising cells which are resistant to induction of apoptosis by inhibitors of FAS. Administration of an inhibitor of FAS according to this invention can induce a cellular response equivalent to a genotoxic stress response in the absence of substantial DNA damage. This invention also provides for use of a FAS inhibitor in the preparation of a medicament for treating a tumor in an individual whose tumor exhibits reduced p53 function.

This application is related to U.S. Provisional Application No. 60/268,680, filed Feb. 15, 2001, which is incorporated herein by reference in its entirety.

### BACKGROUND OF THE INVENTION

1. Field of the Invention

The present invention provides new methods for treating an individual having a tumor. In particular, the method comprises administering to the individual an inhibitor of fatty acid synthase (FAS) in an amount sufficient to retard growth of cells in the tumor.

2. Review of Related Art

Fatty acid synthase (FAS, E.C. 2.3.1.85) is the major biosynthetic enzyme for synthesis of fatty acids from small carbon substrates. FAS is a multi-functional enzyme that performs a repeated sequence of reactions to convert acetyl-CoA and malonyl-CoA to palmitate. Elevated expression of FAS, and abnormally active endogenous fatty acid synthetic metabolism are frequent phenotypic alterations in many human cancers, including carcinomas of breast, prostate, endometrium and colon (Alo, P. L., Visca, P., Marci, A., Mangoni, A., Botti, C., and Di Tondo, U., “Expression of Fatty Acid Synthase (FAS) as a Predictor of Recurrence in Stage I Breast Carcinoma Patients,” Cancer, 77:474-482, (1996); Epstein, J. I., Carmichael, M., and Partin, A. W., OA-519, “(Fatty Acid Synthase) as an Independent Predictor of Pathologic Stage in Adenocarcinoma of the Prostate,” Urology, 45:81-86, (1994); Swinnen, J. V., Esquenet, M., Goossens, K., Heyns, W., and Verhoeven, G., “Androgens Stimulate Fatty Acid Synthase in the Human Prostate Cancer Cell Line LNCAP,” Cancer Research, 57:1086-1090, (1977); Pizer, E., Lax, S., Kuhajda, F., Pasternack, G., and Kurman, R., “Fatty Acid Synthase Expression in Endometrial Carcinoma: Correlation With Cell Proliferation and Hormone Receptors,” Cancer, 83:528-537, (1998a); Rashid, A., Pizer, E. S., Moga, M., Milgraum, L. Z., Zahurak, M., Pasternack, G. R., Kuhajda, F. P., and Hamilton, S. R., “Elevated Expression of Fatty Acid Synthase and Fatty Acid Synthetic Activity in Colorectal Neoplasia,” American Journal of Pathology, 150:201-208, (1997)). The function(s) that active fatty acid synthesis provides for tumor cells appears linked to proliferation, and the bulk of endogenously synthesized fatty acids are incorporated into membrane lipids by proliferating tumor cells (Pizer et al. (1998a); Pizer, E. S., Wood, F. D., Pasternack, G. R., and Kuhajda, F. P., “Fatty Acid Synthase (FAS): a Target for Cytotoxic Antimetabolites in HL60 Promyelocytic Leukemia Cells,” Cancer Research, 56:745-751, (1996a); Jackowski, S., Wang, J., and Baburina, I., “Activity of the Phosphatidylcholine Biosynthetic Pathway Modulates the Distribution of Fatty Acids into Glycerolipids in Proliferating Cells, Biochim Biophys Acta, 1483:301-315, (2000)). Endogenous fatty acid synthetic activity occurs in tumors despite available dietary fatty acid, which down-regulates the pathway in most normal tissues (Weiss, L., Hoffman, G. E., Schreiber, R., Andres, H., Fuchs, E., Korber, E., and Kolb, H. J., “Fatty-Acid Biosynthesis in Man, a Pathway of Minor Importance. Purification, Optimal Assay Conditions, and Organ Distribution of Fatty Acid Synthase”, Biol. Chem. Hoppe-Seyler, 367:905-912, (1986); Ookhtens, M., Kannan, R., Lyon, I., and Baker, N., “Liver and Adipose Tissue Contributions to Newly Formed Fatty Acids in an Ascites Tumor,” Am. J. Physiol., 247:R146-R153, (1984); Sabine, J. R., Abraham, S., and Chaikoff, I. L., “Control of Lipid Metabolism in Hepatomas: Insensitivity of Rate of Fatty Acid and Cholesterol Synthesis by Mouse Hepatoma BW7756 to Fasting and to Feedback Control,” Cancer Research, 27:793-799, (1967)).

The biological basis for this phenotypic alteration is not clear. However, altered fatty acid metabolism represents a novel target for anti-metabolite therapy, since pharmacological inhibition of FAS is selectively cytotoxic for tumor cells, triggering their programmed cell death (Pizer, E. S., Jackisch, C., Wood, F. D., Pasternack, G. R., Davidson, N. E., and Kuhajda, F. P., “Inhibition of Fatty Acid Synthesis Induces Programmed Cell Death in Human Breast Cancer Cells,” Cancer Research, 56:2745-7, (1996b); Pizer, E. S., Wood, F. D., Heine, H. S., Romantsev, F. E., Pasternack, G. R., and Kuhajda, F. P., “Inhibition of Fatty Acid Synthesis Delays Disease Progression in a Xenograft Model of Ovarian Cancer,” Cancer Research, 56:1189-1193, (1996c)). The cytotoxic mechanism of FAS inhibition appears to result from accumulation of the committed substrate, malonyl-CoA, or from related biochemical consequences of inhibition of an active metabolic pathway, since pathway down-regulation before FAS inhibition rescues tumor cell survival (Pizer, E. S., Thupari, J., Han, W. F., Pinn, M. L., Chrest, F. J., Frehywot, G. L., Townsend, C. A., and Kuhajda, F. P., “Malonyl-Coenzyme-A is a Potential Mediator of Cytotoxicity Induced by Fatty-Acid Synthase Inhibition in Human Breast Cancer Cells and Xenografts,” Cancer Research, 60:213-218, (2000)).

Cerulenin, (2R, 3S)-2,3-epoxy-4-oxo-7,10-trans,trans-dodecadienamide, a natural product of Cephalosporium caerulens, is a specific inhibitor of fatty acid synthase enzymes across a broad phylogenetic spectrum (Omura, S., “The Antibiotic Cerulenin, a Novel Tool for Biochemistry as an Inhibitor of Fatty Acid Synthesis,” Bacteriological Reviews, 40:681-697, (1976); Vance, D., Goldberg, I., Mitsuhashi, O., and Bloch, K., “Inhibition of Fatty Acid Synthetases by the Antibiotic Cerulenin,” Biochemical & Biophysical Research Communications, 48:649-656, (1972); Moche, M., Schneider, G., Edwards, P., Dehesh, K., and Lindqvist, Y., “Structure of the Complex Between the Antibiotic Cerulenin and its Target, b-ketoacyl-acyl Carrier Protein Synthase,” J. Biol. Chem., 274:6031-6034, (1999)). Cerulenin irreversibly inhibits FAS by binding covalently to the active site cysteine of the beta keto acyl synthase moiety, which performs the condensation reaction between the elongating fatty acid chain and each successive acetyl or malonyl residue. In Saccharomyces cerevisiae, a point mutation in FAS that confers a 30-fold reduction in affinity of the enzyme for cerulenin also abolishes the drug's growth inhibitory effects accordingly, demonstrating that FAS is a critical target for the drug's cytotoxic effects (Inokoshi, J., Tomoda, H., Hashimoto, H., Watanabe, A., Takeshima, H., and Omura, S., “Cerulenin Resistant Mutants of Saccharomyces cerevisiae with an Altered Fatty Acid Synthase Gene, Mol Gen Genet., 244:90-96, (1994)). A novel small-molecule inhibitor of FAS has recently been syynthesized. It is an α-methylene-γ-butyrolactone with a C7 hydrocarbon side chain, called C-75, with inhibitory effects on fatty acid synthesis comparable to those seen with cerulenin (Kuhajda, F. P., Pizer, E. S., Li, J. N., Mani, N. S., Frehywot, G. L., and Townsend, C. A., “Synthesis and AntiTtumor Activity of a Novel Inhibitor of Fatty Acid Synthase,” Proceedings of the National Academy of Sciences, 97:3450-3454, (2000)).

### SUMMARY OF THE INVENTION

Inhibitors of the enzyme fatty acid synthase (I-FAS) can be used therapeutically to treat cancer cells that overexpress fatty acid synthase (see U.S. Pat. Nos. 5,759,837 and 5,981,575). By administering I-FAS, apoptosis may be induced in malignant cells overexpressing FAS. It has now been discovered that I-FAS may affect cells beyond its ability to induce apoptosis.

In contrast to the previously described apoptosis-inducing therapy, the present invention provides a method of treating malignancies by arresting cell growth. It has now been discovered that the presence of I-FAS impedes progression of cells through the cell cycle. Such an effect is limited to cells undergoing cell division, and therefore inherently avoids toxic effects on mature cells. Treatment of malignancies with I-FAS is broadly applicable for retarding progression of all types of tumors, in addition to eradication of neoplastic cells with p53 mutations and/or FAS overexpression.

The present invention provides new methods for treating an individual having a tumor. In particular, the method comprises administering to the individual an inhibitor of fatty acid synthase (FAS) in an amount sufficient to retard growth of cells in the tumor. In one embodiment, the individual treated by the method of this invention has a tumor comprising cells which do not overexpress FAS and/or the individual has a tumor comprising cells which are resistant to induction of apoptosis by inhibitors of FAS. In a preferred mode, the tumor is malignant. In a particularly preferred mode, the inhibitor of FAS is administered in an amount sufficient to induce a cellular response equivalent to a genotoxic stress response in the absence of substantial DNA damage.

### BRIEF DESCRIPTION OF THE FIGURES

FIG. 1 shows DNA content of RKO cells analyzed by flow cytometry after various time periods of exposure to cerulenin (10 μg/mL).

FIG. 2A shows bromodeoxyuridine (BrdU) pulse/chase analysis of pulse labeled RKO cells chased for various time periods in the absence of FAS inhibitors.

FIG. 2B shows BrdU pulse/chase analysis of pulse labeled RKO cells chased for various time periods in the presence of cerulenin (10 μg/mL).

FIG. 3 shows cyclin A- and cyclin B1-associated kinase activities which were determined by an immunocomplex-kinase assay after RKO cells were exposed to FAS inhibitors for the indicated time periods. FAS inhibition induces a marked reduction of S- and G2/M-associated cdk activity during the early period of exposure.

FIG. 4. shows accumulation of p53 and p21 induced in RKO colon carcinoma cells by pharmacological inhibitors of FAS. Cells were treated with cerulenin (10 μg/ml) (A) or C-75 (10 μg/ml) (B) for the stated exposure times, and analyzed by immunoblotting for p53 and p21 protein content, with actin as an internal control.

FIG. 5. shows cerulenin- or C-75-treated MCF7 breast carcinoma cells subjected to alkaline single cell gel electrophoresis (comet assay). Olive tail moment indicates electrophoretic mobility of DNA induced by DNA damage.

FIG. 6 shows RKO cells without or with a stably-transfected dominant negative mutant p53 gene which were subjected to multi-parameter flow cytometry after 24 h of exposure to cerulenin. Ungated two-dimensional analysis of DNA content versus MC540 fluorescence is displayed after no drug (A and B), cerulenin (5 μg/ml) (C and D), and cerulenin (10 μg/ml) (E and F). Apoptotic and non-apoptotic cells are in upper and lower boxes, respectively.

FIG. 7. shows constitutive fatty acid synthesis pathway activity of parental and p53 deficient lines are similar (A). Cerulenin, C-75 and TOFA inhibit fatty acid synthesis to 60% or less of control levels at the doses used [μg/ml] (B). Apoptotic fraction of colon and breast carcinoma cells after 24 h exposure to FAS inhibitors, analyzed as in FIG. 6 (C and E). Parallel determinations of sensitivity to FAS inhibitors were performed by clonogenic assay after a 6-h drug exposure. (D and F). SW480 is a colon carcinoma line with a naturally-occurring p53 mutation. SKBr3 is a breast carcinoma line with a naturally-occurring p53 mutation.

FIG. 8 shows DNA content of RKO cells exposed to [cerulenin,10 μg/ml] or [C-75,10 μg/ml] for the indicated times, without or with 1 hour pretreatment with TOFA (5 μg/ml to inhibit malonyl-CoA synthesis). FAS inhibitors (cerulenin or C-75) induced growth arrest independent of malonyl-CoA accumulation.

FIG. 9 shows a comparison of FAS enzyme levels in non-transformed human cell line, IMR-90, and a panel of tumor lines.

### DETAILED DESCRIPTION OF THE EMBODIMENTS

In previous inventions, cancer cells with high levels of fatty acid synthase (FAS) and fatty acid synthesis were shown to undergo apoptosis when treated with inhibitors of (FAS) (U.S. Pat. No. 5,759,837; U.S. Pat. No. 5,981,575). The present invention demonstrates that cancer cells with low levels of fatty acid synthase (FAS) and fatty acid synthesis, and intact p53 signaling, undergo growth arrest when treated with inhibitors of FAS, whereas those with loss of p53 function, undergo rapid, extensive apoptosis. A summary of the effect of FAS inhibitors on cells with varying levels of FAS expression and p53 function is shown in the accompanying Table.

TABLE
Effect of FAS Inhibitors on Tumor Cells

| | High FAS Expression | Low FAS Expression |
|---|---|---|
| intact p53 | apoptosis | growth arrest |
| reduced p53 function | apoptosis | apoptosis |

This invention provides a rationale to treat patients with inhibitors of FAS regardless of the rate of fatty acid synthesis or level of FAS expression, and it shows that FAS inhibitor therapy may be effective against the most virulent and treatment resistant human cancers that characteristically have reduced or absent p53 function.

This invention describes a novel, anti-tumor effect of FAS inhibitors in human cancer, namely growth inhibition. As disclosed herein, FAS inhibitors have anti-tumor effect regardless of the level of FAS expression or rate of fatty acid synthesis. Furthermore, this invention links p53 function to fatty acid synthesis perturbation in cancer cells; cancer cells with dysfunctional p53 signaling undergo apoptosis when treated with FAS inhibitors. It is also disclosed that growth inhibition induced by FAS inhibition is not dependent upon malonyl-CoA accumulation, but rather from lipid product depletion.

As disclosed herein, FAS inhibition has an anti-tumor activity in human cancer cells regardless of the level of FAS expression or fatty acid synthesis activity. All human tumors may respond to FAS inhibitor therapy. This increases the scope of FAS inhibitor therapy from patients whose tumors have high levels of fatty acid synthesis to all patients. The subset of human tumors with high levels of fatty acid synthesis and/or loss of p53 function will have a cytotoxic, apoptotic response to FAS inhibition. The subset of human tumors with low levels of fatty acid synthesis and intact p53 function will have a cytostatic response to FAS inhibition.

Cells having low levels of fatty acid synthesis can be identified by immunoblotting using an antibody specific for FAS to develop the blot. Such antibodies are disclosed in U.S. Pat. No. 5,872,217, incorporated herein by reference. Typically, such cells have FAS levels equal to or lower than the level of FAS detected by immunoblot in RKO cells (see Example 9). Alternatively, FAS levels may be characterized by comparison to IMR-90 cells, which express FAS at a level about four-fold lower than RKO cells. IMR-90 cells may be obtained from the American Type Culture Collection, Manassas, Va., USA, where they have been deposited under ATCC Accession No. ______.

An FAS inhibitor (I-FAS) is a compound that specifically interferes with the enzymatic activity of fatty acid synthase (FAS). The inhibition may be determined by carrying out FAS assays in the presence and absence of the compound suspected to be an inhibitor. Suitable assays are described in the Examples, although the skilled artisan could readily devise alternative assays. FAS inhibitors according to this invention are specific in that they do not indiscriminately directly inhibit the activities of other enzymes, although cross-inhibition of a few related enzymes is not outside the contemplation of this invention. The skilled artisan will recognize that pleiotropic effects of I-FAS on down-stream or ancillary pathways is to be expected. Exemplary compounds having the characteristics of I-FAS according to this invention include the antibiotic cerulenin and the novel compound C-75, as well as other compounds disclosed in U.S. Pat. Nos. 5,759,837 and 5,981,575, incorporated herein by reference. The skilled artisan can readily determine whether a particular compound is an I-FAS.

Cells which over-express FAS are described in U.S. Pat. No. 5,759,837, incorporated herein by reference. FAS is normally expressed in the liver and in adipose tissue, where it functions to convert dietary carbohydrate to fat, and in some specialized contexts, like lactating breast and the surfactant producing cells of the lung, but has little expression in most other normal adult tissues which predominantly utilize circulating sources of fatty acids. Detection of FAS expression in tissues that normally do not express it, by detecting mRNA encoding FAS or by detecting fatty acid synthesis in the cell (as described below in the Examples), is an indication that the cells expressing FAS may not be completely normal.

Genotoxic type stress response is a set of cellular events which mimic events that occur in cells containing damaged DNA. It is well established that DNA damage (for example, due to radiation) leads to growth arrest and accumulation of cells in G₁ and G₂/M. The genotoxic type stress response disclosed herein produces these cellular manifestations in cells without sufficient DNA damage to trigger the response.

Individuals that may be treated by the methods of this invention include animals, particularly mammals, more particularly humans. Typically, these individuals will be tumor bearing, and the tumors may be malignant or benign. While treatment of tumors with I-FAS was taught for tumors containing cells that overexpress FAS in U.S. Pat. Nos. 5,759,837 and 5,981,575, the present invention is generally concerned with individuals bearing tumors with cells that do not overexpress FAS. Formulation and administration of I-FAS to such individuals will be analogous to that described in the cited patents.

In order to gain further insight into the biological role of the fatty acid synthetic pathway for tumor cells, and the nature of the growth inhibition resulting from inhibition of FAS, the present inventors examined the cellular events that follow inhibition of FAS and precede cell death. Two chemically distinct inhibitors of FAS were studied in parallel to provide a generic picture of the consequences of loss of FAS function. FAS inhibitors produce rapid, profound blocks of DNA replication and S-phase progression in human cancer cells (Pizer, E. S., Chrest, F. J., DiGiuseppe, J. A., and Han, W. F., “Pharmacological Inhibitors of Mammalian Fatty Acid Synthase Suppress DNA Replication and Induce Apoptosis in Tumor Cell Lines,” Cancer Research, 58:4611-4615, (1998b)). Fatty acid synthesis inhibition occurred within 30 min and DNA synthesis inhibition occurred within 90 min of drug exposure, and induction of apoptosis followed several hours later. The suppressive effect of fatty acid synthesis inhibition on DNA replication was indirect, because expression of certain viral oncogenes alleviated it. The inventors further characterized the cellular response to FAS inhibition.

RKO colon carcinoma cells were selected for study because they undergo little apoptosis within the first 24 h after FAS inhibition. Instead, RKO cells exhibited a bi-phasic stress response, with a transient accumulation in S and G2 at 4 and 8 h that corresponds to a marked reduction in cyclin A- and B1-associated kinase activities, followed by accumulation of p53 and p21 proteins at 16 and 24 h, and growth arrest in G1 and G2. RKO cells stress response was marked by early loss of S phase and G2 cyclin-dependent kinase activity, and subsequent accumulation of p53 and p21 proteins may protect RKO cells from the cytotoxic effects of FAS inhibition. The delays in cell cycle progression with redistribution of cells into G1 and G2 after FAS inhibition were suggestive of cell cycle checkpoint activation by the tumor suppressor p53, as occurs after genotoxic or other cellular stresses (Meek, D. W., “Post-Translational Modification of p53 and the Integration of Stress Signals,” Pathol Biol., 45:804-814, (1997)). While the response of RKO cells to FAS inhibition resembled a genotoxic stress response, but DNA damage did not appear to be an important downstream effect of FAS inhibition, since none was detected using the single cell gel electrophoresis assay (comet assay) to assess DNA damage.

Cell cycle progression is regulated through the sequential activation and inactivation of cyclin-dependent kinases (cdks) that, in turn, phosphorylate key regulatory proteins (Pines, J., “Cyclins and Cyclin-Dependent Kinases: Theme and Variations,” Advances in Cancer Research, 66:181-212, (1995)). Cyclin A/cdk2 complex activity is required for efficient DNA replication, and the activity of complexes containing cdc2 and cyclins A and the B is required for passage through G2 and mitosis. FAS inhibitors induce inhibition of S phase and G2 cyclin-dependent kinase activity during the early period of exposure.

Inhibition of FAS induced p53 and p21 protein accumulation and G1/G2 redistribution in RKO cells, which have an intact p53 pathway (and in other cell lines with wild type p53, not shown). However, many tumor lines with p53 mutations undergo apoptosis within 24 h of exposure to FAS inhibitors (Pizer et al., 1998b). The inventors determined the effect of p53 function on survival after FAS inhibition by comparing two pairs of isogenic cell lines with wild-type and altered p53 function. P53 function is probably important in protecting RKO cells from FAS inhibition, because RKO cells expressing a dominant negative mutant p53 gene underwent extensive apoptosis within 24 h after FAS inhibition, similar to many other tumor lines. Loss of p53 function substantially increased the sensitivity of tumor cells to FAS inhibitors. Sensitization of cells to FAS inhibitors by loss of p53 raises the possibility that these agents may be clinically useful against malignancies carrying p53 mutations.

Accumulation of malonyl-CoA, the committed substrate for fatty acid synthesis, is likely to participate in the cytotoxicity of FAS inhibition, since down regulation of malonyl-CoA production alleviated the toxicity of cerulenin and C-75, and substantially reduced the apoptotic fraction at 24 hours (Pizer et al, 2000). However, while induction of apoptosis appeared related to accumulation of the substrate, malonyl-CoA, after FAS inhibition, the cytostatic effects were independent of malonyl-CoA accumulation, and may have resulted from product depletion.

Growth Arrest due to Lipid Product Depletion.

Although not wishing to be bound by any particular mechanism, the inventors note that the bi-phasic stress response to FAS inhibition may result from lipid product depletion. The kinetics of the response of RKO cells to FAS inhibition illustrated in Examples 1 through 4 below suggests a rapid onset of a stress response. This response is characterized by a marked reduction in cyclin A- and B-associated kinase activities, an early suppression of DNA replication and an accumulation of cells in the S and G2 phases during the first 8 h of drug exposure, followed by enhanced expression of p53 and p21 proteins and growth arrest in G1 and G2 by 16 and 24 h.

While malonyl-CoA accumulation appears involved in triggering apoptosis after FAS inhibition, the growth arrest stress response produced by FAS inhibition may be due to altered lipid production, since ACC inhibition did not relieve it. Most of the fatty acids produced by tumor cells are incorporated into membrane phospholipids, and phospholipid synthesis is inhibited when fatty acid synthesis is inhibited (Pizer et al., 1996a; Jackowski et al., 2000). Phospholipid biosynthesis is greatest during the G1 and S phases, with doubling of the membrane mass occurring during S phase to prepare for cell division (Jackowski, S., Coordination of Membrane Phospholipid Synthesis with the Cell Cycle,” Journal of Biological Chemistry, 269:3858-3867, (1994)). It is possible, therefore, that limitation of phospholipid synthesis during the S phase affects DNA replication, or independently triggers late cell cycle delays similar to the pre-mitotic checkpoints of yeast (Thuriaux, P., Nurse, P., and Carter, B., “Mutants Altered in the Control Co-Ordinating Cell Division With Cell Growth in the Fission Yeast Schizosaccharomyces pombe,” Mol Gen Genet., 161:215-220, (1978); Enoch, T. and Nurse, P., “Mutation of Fission Yeast Cell Cycle Control Genes Abolishes Dependence of Mitosis on DNA Replication,” Cell, 60:665-673, (1990)).

Notably, two ether lipids that specifically inhibit the CTP:phosphocholine cytidylyltransferase, an important enzyme in phospholipid synthesis, produce similar G2/M delays and are selectively cytotoxic to transformed cells (Boggs, K., Rock, C. O., and Jackowski, S., “The Antiproliferative Effect of Hexadecylphosphocholine Toward HL60 Cells is Prevented by Exogenous Lysophosphatidylcholine,” Biochimica et Biophysica Acta., 1389:1-12, (1998)). Studies in lower eukaryotes and prokaryotes have shown a requirement for active fatty acid synthesis at the time of cell division, either for simple mitosis, or for sporulation (Saitoh, S., Takahashi, K., Nabeshima, K., Yamashita, Y., Nakaseko, Y., Hirata, A., and Yanagida, M., “Aberrant Mitosis in Fission Yeast Mutants Defective in Fatty Acid Synthetase and Acetyl CoA Carboxylase,” Journal of Cell Biology, 134:949-961, (1996)). The defects in these systems appear related to chromatin configuration, or to transcriptional activation of key genes. In the FAS inhibition system discussed here, however, the specific mechanisms whereby cyclin A- and B-associated kinase activities decrease in RKO cells remain to be studied in detail.

Role of Tumor Suppressor p53 in the Response to FAS Inhibitors.

The observation that FAS inhibitors induced the accumulation of p53 and p21 proteins might suggest that DNA damage is occurring, either as a direct effect of the drugs on the DNA molecule, or as a downstream effect of FAS inhibition. However, several other observations argue against DNA damage. First, the toxic effect of cerulenin was found to be dependent on its ability to inhibit FAS in yeast, thus ruling out a significant direct effect of cerulenin on DNA (Inokoshi et al., 1994). Secondly, toxicity in tumor cells is modulated by alterations in activity of the fatty acid synthesis pathway and substrate levels. Finally, no DNA damage was detected using the single cell gel electrophoresis (comet) screening assay, an assay that has been shown to be very sensitive in detecting low levels of DNA damage. Consistent with these observations, no differences were detected in the sensitivity to FAS inhibitors of cells deficient in ATM (mutated in ataxia telangectasia) versus controls.

While the first and most extensively studied function described for the tumor suppressor protein p53 was the induction of growth arrest and apoptosis after DNA damage (Kuerbitz, S. J., Plunkett, B. S., Walsh, W. V., and Kastan, M. B., “Wild Type p53 is a Cell Cycle Checkpoint Determinant Following Irradiation,” Proceedings of the National Academy of Sciences of the United States of America, 89:7491-7495, (1992); Agarwal, M. L., Taylor, W. R., Chernov, M. V., Chernova, O. B., and Stark, G. R., “The p53 Network,” Journal of Biological Chemistry, 273:1-4, (1998); Magnelli, L., Ruggiero, M., and Chiarugi, V., “The Old and the New in p53 Functional Regulation,” Biochemical & Molecular Medicine, 62:3-10, (1997); Smith, M. L. and Fornace, A. J., Jr., :p53-Mediated Protective Responses to UV Irradiation,” Proceedings of the National Academy of Sciences of the United States of America, 94:12255-12257, (1997)), more recently, important roles for p53 have been recognized in the cellular responses to a variety of non-genotoxic metabolic stresses, including hypoxia, acidosis, and perturbations of RNA and protein synthesis (Linke, S. P., Clarkin, K. C., Di Leonardo, A., Tsou, A., and Wahl, G. M., “A Reversible, p53-Dependent G0/G1 Cell Cycle Arrest Induced by Ribonucleotide Depletion in the Absence of Detectable DNA Damage,” Genes Dev., 10:934-947, (1996); Schmaltz, C., Hardenbergh, P. H., Wells, A., and Fisher, D. E., “Regulation of Proliferation-Survival Decisions During Tumor Cell Hypoxia,” Molecular & Cellular Biology, 18:2845-2854, (1998); An, W. G., Kanekal, M., Simon, M. C., Maltepe, E., Blagosklonny, M. V., and Neckers, L. M., “Stabilization of Wild-Type p53 by Hypoxia-Inducible Factor 1alpha,” Nature, 392:405-408, (1998); Graeber, T. G., Osmanian, C., Jacks, T., Housman, D. E., Koch, C. J., Lowe, S. W., and Giaccia, A. J., “Hypoxia-Mediated Selection of Cells with Diminished Apoptotic Potential in Solid Tumours,” Nature, 379:88-91, (1996); Alessenko, A. V., Boikov, P., Filippova, G. N., Khrenov, A. V., Loginov, A. S., and Makarieva, E. D., “Mechanisms of Cycloheximide-Induced Apoptosis in Liver Cells,” FEBS Letters, 416:113-116, (1997); Pritchard, D. M., Watson, A. J., Potten, C. S., Jackman, A. L., and Hickman, J. A., Inhibition by Uridine But Not Thymidine of p53-Dependent Intestinal Apoptosis Initiated by 5-Fluorouracil: Evidence for the Involvement of RNA Perturbation,” Proceedings of the National Academy of Sciences of the United States of America, 94:1795-1799, (1997)). The current study indicates that perturbation of fatty acid synthesis also belongs on the list of metabolic stresses regulated by p53.

Whether the effects of FAS inhibition are observed as apoptosis or growth arrest clearly is influenced by p53 function. Since constitutive fatty acid synthesis activity, and inhibitor effects were similar between the paired parental and p53 deficient cells, it is unlikely that levels of malonyl-CoA accumulation were substantially different, however, the ability of the cell to survive malonyl-CoA accumulation may be greater in cells with intact p53. The relatively low fatty acid synthesis pathway activity of RKO cells (less malonyl-CoA) combined with intact p53 function may underlie the minimal apoptosis produced by FAS inhibitors in RKO cells, and in various non-transformed cells. It is likely that induction of p21 promotes growth arrest and exerts a protective effect after FAS inhibition, as it has been shown to do in a variety of other stress paradigms (Gorospe M, W. X., Holbrook N J, “Functional Role of p21 During the Cellular Response to Stress,” GeneExpression, 7:377-385, (1999)).The triggering of apoptosis after FAS inhibition is very rapid, and probably occurs before p21 induction. FAS inhibitors triggered comparable apoptotic responses in the majority of tumor lines with mutant p53 status that have been studied. The predominant pattern of sensitization by loss of p53 function suggests that endogenous fatty acid synthesis will hold special appeal as an experimental therapeutic target. FAS inhibitors combine the target specificity for cancer cells afforded by both elevated fatty acid synthesis and loss of p53 function.

### EXAMPLES

### Example 1

### FAS Inhibitors Induce Delays in Cell Cycle Progression as Shown by DNA Content of Treated Cells

Investigation by flow cytometric analysis of serial samples taken after FAS inhibition demonstrated a bi-phasic effect on the cell cycle progression of RKO colon carcinoma cells. Cells were cultured in DMEM with 10% fetal bovine serum (Hyclone). Cells were screened periodically for mycoplasma contamination (Gen-probe). Cerulenin (Sigma) C-75 and TOFA, dissolved in DMSO, were added from 5 mg/ml stock solutions; the final concentration of DMSO in cultures was at or below 0.2%. Cells were exposed to cerulenin or C-75 for the indicated doses and time intervals, then detached from plastic with trypsin for flow cytometry analysis. DNA content was measured by multiparameter flow cytometry using a FACStar^{Plus} flow cytometer equipped with argon and krypton lasers (Becton Dickinson).

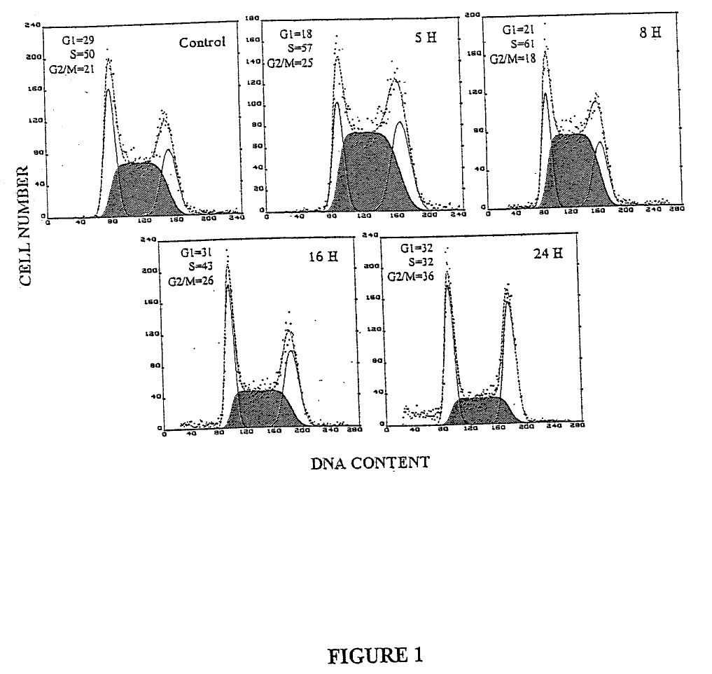

When proliferating cells were exposed to 10 μg/ml cerulenin, there was a redistribution of cells into S phase and G2/M during the early time points, at 5 and 8 h, compatible with inhibited progression through these cell cycle phases (FIG. 1). Later, at 16 and 24 h, the S-phase fraction decreased substantially, with a redistribution of cells into G1 and G2/M. This effect was characteristic of both cerulenin and C-75 treatment on RKO cells, as well as on other cell lines that had limited apoptotic responses to FAS inhibitors (not shown).

### Example 2

### Delays Induced in Cell Cycle Progression by FAS Inhibitors

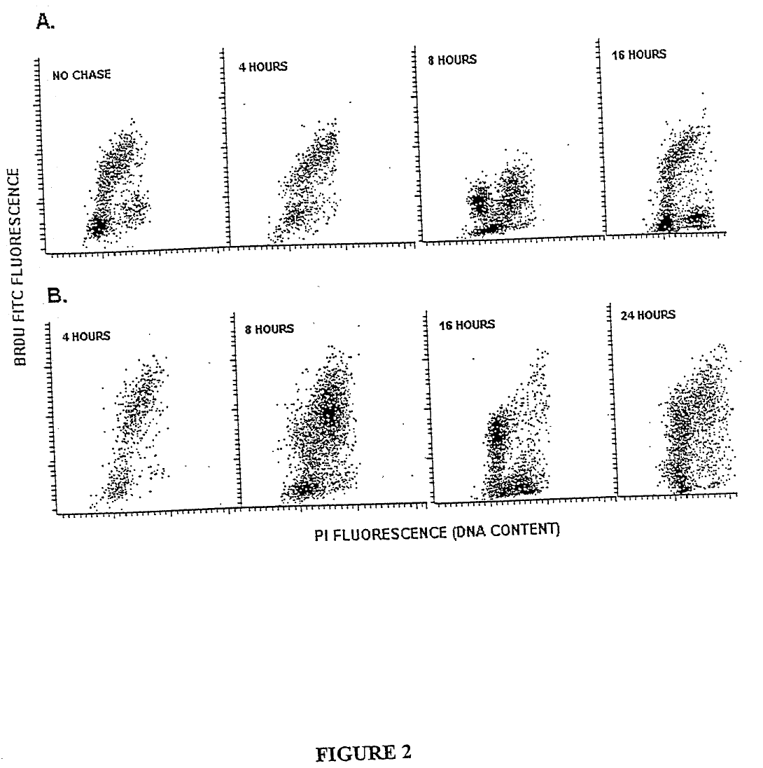

A similar experiment measured cell cycle progression by pulse/chase labeling with bromodeoxyuridine (BrdU, FIG. 2). The BrdU-positive S-phase fraction at time zero progresses through the cell cycle at later time points. B. The progress of BrdU pulse-labeled RKO cells through the cell cycle was monitored over 24 h without inhibition of FAS, or during exposure to cerulenin (10 μg/ml) or C-75 (10 μg/ml).

Bromodeoxyuridine Detection by Laser Scanning Cytometry:

Dual-parameter detection of Bromodeoxyuridine (BrdU) labeling and DNA content was performed using a laser scanning cytometer (Compucyte Corp.). Cell cultures were pulse-labeled for 20 min with 10 μM BrdU, and chased for the indicated times in the absence or presence of drug, then detached from plastic with trypsin, ethanol-fixed and applied to glass slides. Cells were subjected to standard heat-induced epitope retrieval (DAKO) before staining with anti-BrdU antibody (DAKO) and FITC-conjugated goat anti-mouse antibody (CALTAG, DAKO Autostainer™). DNA content was assessed after staining with 0.5% propidium iodide. Data were collected and analyzed using WinCyte software (Compucyte Corp.).

A 20-min exposure of proliferating cells to BrdU labeled the S-phase population at time zero. Chase samples were collected at 4, 8, 16 and 24 h. In control cultures (FIG. 2A), the BrdU-labeled population progressed through G2/M, first reappearing in the G1 population in the 8 h chase sample. By 16 h the BrdU-labeled population was in G1 and S phase again, indicating a complete cell cycle traverse time of approximately 16 h for RKO cells. By 24 h labeled and unlabeled populations were distributed throughout the cell cycle, indicating continued progression and loss of synchronization (not shown).

RKO cells treated with FAS inhibitors demonstrated substantial delays in cell cycle progression that corresponded with the flow cytometry single-parameter cell cycle results (FIG. 1). The cerulenin-treated samples are shown in FIG. 2B; C-75-treated populations exhibited a similar response (not shown). The treated 8-h chase sample showed no BrdU-labeled cells yet reappearing in G1, in agreement with our observation that cells redistribute into the S and G2/M phases seen in FIG. 1. By 16 h, most of the BrdU-labeled cells had reentered G1, but very few had entered S phase, and by 24 h most cells, labeled and unlabeled, were in G1 or G2/M, and were still synchronized, indicating that cell cycle progression had slowed down substantially.

### Example 3

### FAS Inhibition Induces a Marked Reduction of S- and G2/M-Associated cdk Activity During the Early Period of Exposure

The effect of FAS inhibitors on the activity of cyclin/cdk complexes in RKO cells was determined in a time course analysis. After RKO cells were exposed to FAS inhibitors for the indicated time periods, cyclin A- and cyclin B 1-associated kinase activities were determined by an immunocomplex-kinase assay.

Immunoprecipitation and Immunocomplex-Kinase Assay:

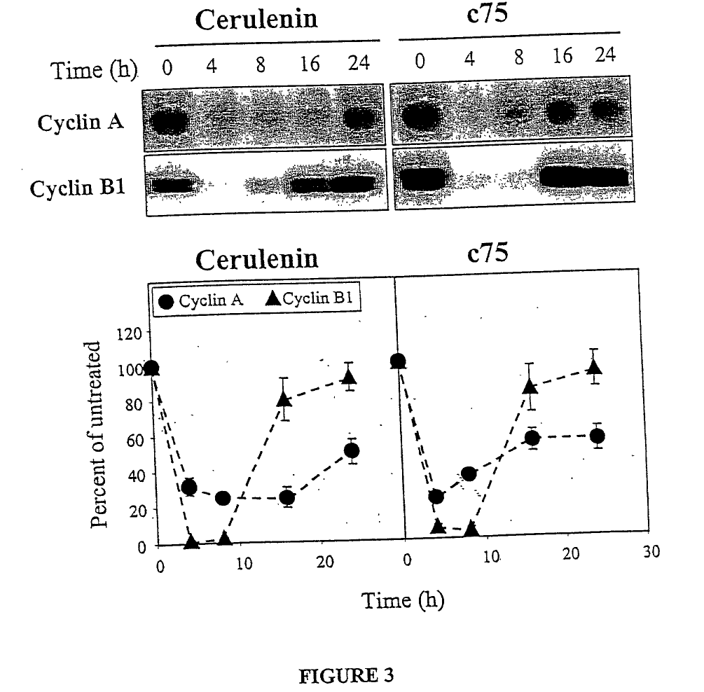

Five×10⁶ RKO cells per 100-mm plate were treated with 10 μg/ml cerulenin or C-75 for the indicated time intervals. The control cells received equivalent amounts of DMSO. After drug treatment, the plates were washed once and lysed with immunoprecipitation (IP) buffer (150 mM Tris, pH 7.4, 150 mM NaCl, 1% Triton X-100, 0.5% NP-40, 1 mM ethyleneglyco-bis-tetraacetic acid, 0.2 mM sodium vanadate, and 0.2 mM phenylmethylsulfonyl fluoride). Protein concentration was measured using the BCA Protein Assay Kit (Pierce). One hundred μg of protein from each sample was incubated at 4° C. for 1 h with 1 μg of primary antibody (anti-human cyclin A rabbit polyclonal antibody or anti-human cyclin B 1 monoclonal antibody, Santa Cruz) and then overnight after addition of Protein A or protein G-Sepharose (Santa Cruz). The immunoprecipitates were washed twice with IP buffer and once with kinase buffer (10 mM Tris, pH 7.4, 150 mM NaCl, 10 mM MgCl₂ and 0.5 mM DTT) and resuspended in 40 μl of kinase buffer containing 1 μg of histone H1, 25 μM of ATP, and 2.5 μCi of γ-³²P-ATP. Following a 30-min incubation at 30° C., the reaction was terminated by adding 40 μl of 2× Laemmli sample buffer. Samples were resolved by electrophoresis through 12% SDS-polyacrylamide gels and quantitated on a Storm 820 system (Molecular Dynamics). All samples were run in duplicate, and each experiment was performed at least twice. Means and standard errors of one representative experiment are shown in FIG. 3B.

As shown (FIG. 3), the kinase activity associated with immunoprecipitated complexes containing cyclin A decreased to less than 40% of control levels at 4 and 8 h after exposure to either cerulenin or C-75, then increased moderately at later time points. The kinase activity associated with immunoprecipitated cyclin B decreased to less than 5% of control levels by 4 and 8 h after exposure to either cerulenin or C-75, then increased to greater than 80% of control levels at 16 and 24 h. These changes in S and G2 cdk activity correlated well with the bi-phasic pattern of cell cycle distribution demonstrated in FIGS. 1 and 2. Immunoblots of cyclin A and B levels performed in parallel with the experiment in FIG. 3, demonstrate that unlike the associated kinase activities, the cyclin levels do not decrease until 24 h (not shown).

### Example 4

### Accumulation of p53 and p21 is Induced in RKO Colon Carcinoma Cells by Pharmacological Inhibitors of FAS

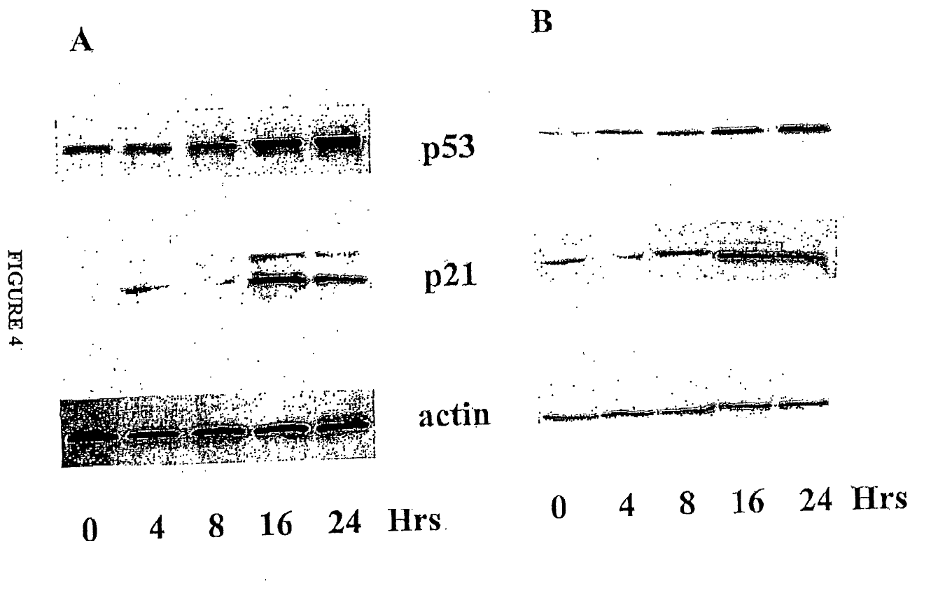

Accumulation of p53 protein, and the p53-regulated cdk inhibitor p21WAF1/CIP1, were assayed by immunoblotting in a parallel time course after inhibition of FAS (FIG. 4). Cells were treated with cerulenin (10 μg/ml) (A) or C-75 (10 μg/ml) (B) for the stated exposure times, and analyzed by immunoblotting for p53 and p21 protein content, with actin as an internal control. One million cells per 60-mm plate were treated with 10 μg/ml cerulenin or C-75 in duplicate for the indicated time intervals; control cells received equivalent amounts of DMSO. After drug treatment, cells were lysed with 200 μl Laemmli sample buffer and boiled. Ten μl of each lysate per lane was separated by SDS-PAGE, transferred to nitrocellulose, and exposed to antibodies against p53 (Pab1801, Oncogene Research Products), p21 (6B6, PharMingen) or actin (I-19, Santa Cruz), followed by horseradish peroxidase-conjugated goat anti-mouse or rabbit anti-goat antibody (Pierce), enhanced chemiluminescence (Amersham) and autoradiography.

p53 and p21 protein levels were unchanged or decreased during the early period of FAS inhibitor exposure. However, treatment with 10 μg/ml of either cerulenin or C-75 induced accumulation of p53 and p21 protein at 16 and 24 h in RKO cells (FIG. 4). Of note, p21 mRNA levels did not show increases of the same magnitude, suggesting translational and/or post-translational mechanism(s) regulating p21 accumulation (not shown).

### Example 5

### FAS Inhibitors do not Induce DNA Damage

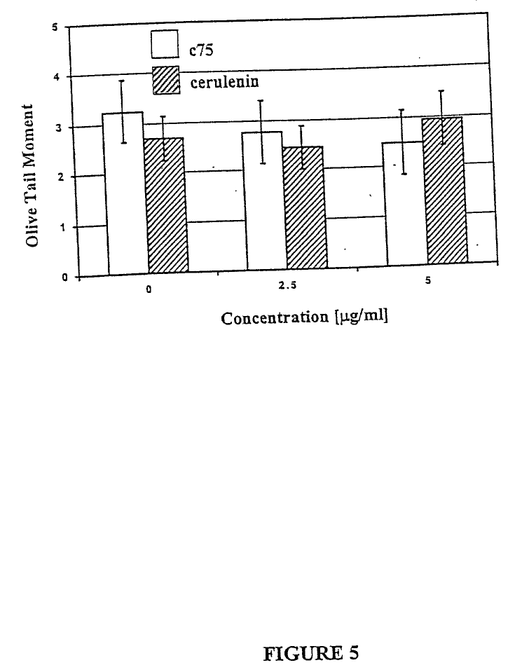

To determine whether significant DNA damage occurred after FAS inhibitor exposure, alkaline single cell gel electrophoresis (comet assay) was performed on MCF7 breast cancer cells after exposure to concentrations of cerulenin and C-75 which resulted in 75% survival (FIG. 5). This assay detects DNA strand breaks, and a spectrum of alkali-labile DNA damage at low levels (Singh, N. P., McCoy, M. T., Tice, R. R., and Schneider, E. L., “A simple Technique for Quantitation of Low Levels of DNA Damage in Individual Cells,” Experimental Cell Research, 175:184-191, (1988); Plappert, U., Raddatz, K., Roth, S., and Fliedner, T. M., “DNA-Damage Detection in Man After Radiation Exposure—the Comet Assay—Its Possible Application for Human Biomonitoring,” Stem Cells, 13 Supplement 1:215-222, (1995)).

Cerulenin- or C-75-treated MCF7 breast carcinoma cells were subjected to alkaline single cell gel electrophoresis (comet assay). MCF7 breast cancer cells were treated with cerulenin or C-75 for 3 h at doses bracketing 75% survival at 24 h. All experiments were repeated three times and duplicate slides from each experiment were prepared and scored. The comet assay was performed under alkaline conditions, essentially as described (Singh et al., 1988), with some modifications. In brief, cells were suspended in 0.5% low melting point agarose (LMA) (Trevigen) and spread on glass microscope slides precoated with 1% normal agarose. After immersion in lysis solution (Trevigen) at 4° C. for a minimum period of 1 h to remove cellular proteins, the slides were immersed in electrophoresis buffer (300 mM NaOH, 1 mM EDTA, pH>13) for unwinding DNA, and subjected to electrophoresis (25 V, 300 mA) for 20 min. Neutralized, dehydrated slides were stained with ethidium bromide (2 ng/ml) and comets scored under a Nikon fluorescence microscope (with TRITC filters) coupled to a KOMET 4.0 software (Kinetic Imaging Ltd).

Olive tail moment indicates electrophoretic mobility of DNA induced by DNA damage. The comet parameters, ‘Olive Tail Moment’ (OTM), ‘Tail Length’ (DNA migration) and ‘percentage DNA in the tail’ were used as indicators of DNA damage. One hundred consecutive cells were scored from the middle of each slide, and the means calculated. The final results were expressed as the (mean of the individual means)±(standard deviation of the means). Lymphoblasts exposed to 0 or 1 Gy gamma irradiation had olive tail moments of 0.9±0.3 and 7.1±0.8 in this experiment. Exposure to 5 cGy gamma irradiation typically produces an olive tail moment of twice the control.

Neither cerulenin nor C-75 induced olive tail moments over background values for untreated control cells, indicating that DNA damage was not induced by either agent at doses previously shown to induce inhibition of DNA synthesis and reduce clonogenic activity (Pizer et al., 2000; Pizer et al., 1998b). This suggests that C-75 and cerulenin induced cytotoxic, not genotoxic damage to cells in an assay that under similar conditions readily detected DNA damage induced by 5 cGy of gamma irradiation or 25 μm H₂O₂. A similar absence of DNA damage was seen after drug treatment of GM1310B human lymphoblasts (not shown).

### Example 6

### Loss of p53 Function Substantially Increased the Sensitivity of Tumor Cells to FAS Inhibitors

The effect of p53 function on survival after FAS inhibition was investigated by comparing two pairs of isogenic cell lines with wild-type and altered p53 function. RKO cells were rendered p53-mutant by stable transfection with a dominant-negative mutant p53 gene (RKO-p53); the human breast carcinoma cell line MCF7 was rendered p53-deficient by constitutive expression of the human papilloma virus 16 E6 gene (MCF7-E6) (Fan, S., Smith, M. L., Rivet, D. J., 2nd, Duba, D., Zhan, Q., Kohn, K. W., Fornace, A. J., Jr., and PM, O. C., “Disruption of p53 Function Sensitizes Breast Cancer MCF-7 Cells to Cisplatin and Pentoxifylline,” Cancer Research, 55:1649-1654, (1995)).

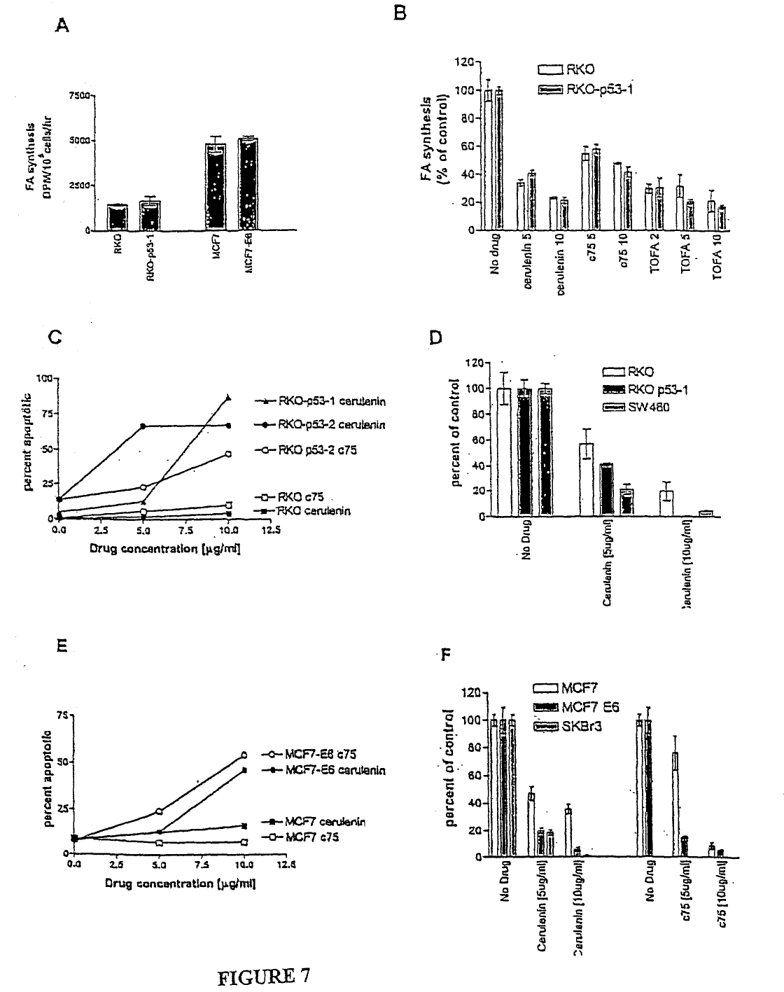

Fatty acid synthesis was compared in cells were plated at 5×10⁴/well in 1 ml in 24 well plates and incubated overnight. Fatty acid synthesis was assayed with a 2 hour pulse of [U-¹⁴C]-acetic acid, 1 μCi/well, followed by Folch extraction and scintillation counting (Pizer et al., 1996a). The fatty acid synthetic pathway activity in these paired lines was very similar, so loss of p53 function had no discernable effect on fatty acid synthesis level (FIG. 7A). For determination of residual pathway activity after FAS inhibitor exposure (FIG. 7B) a 3 hour pulse of [U-¹⁴C]-acetic acid, 1 μCi/well, was performed after 2 hours of drug exposure. All determinations were in triplicate. Data are presented as mean values with bars showing the standard error. Calculations and graphing were performed in Prism 2.0 (GraphPad). Cerulenin, C-75 and TOFA inhibit fatty acid synthesis to 60% or less of control levels a the doses used ((Pizer et al., 2000; Pizer et al., 1998b), see also FIG. 7B). FAS inhibitors produced comparable reduction of pathway activity in the paired lines (FIG. 7B).

Cells were exposed to cerulenin or C-75 for the indicated doses and time intervals, then detached from plastic with trypsin for flow cytometry analysis. Apoptosis was measured by multiparameter flow cytometry using a FACStar^{Plus} flow cytometer equipped with argon and krypton lasers (Becton Dickinson). Apoptosis was quantified using 10 μg/ml merocyanine 540 (Sigma), which detects altered plasma membrane phospholipid packing that occurs early in apoptosis (Pizer et al., 1998b; Reid, S., Cross, R., and Snow, E. C., “Combined Hoechst 33342 and merocyanine 540 Staining to Examine Murine B Cell Cycle Stage, Viability and Apoptosis,” Journal of Immunological Methods, 192:43-54, (1996); Mower, D. A., Jr., Peckham, D. W., Illera, V. A., Fishbaugh, J. K., Stunz, L. L., and Ashman, R. F., “Decreased Membrane Phospholipid Packing and Decreased Cell Size Precede DNA Cleavage in Mature Mouse B Cell Apoptosis,” Journal of Immunology, 152:4832-4842, (1994); Castedo, M., Hirsch, T., Susin, S. A., Zamzami, N., Marchetti, P., Macho, A., and Kroemer, G., Sequential Acquisition of Mitochondrial and Plasma Membrane Alterations During Early Lymphocyte Apoptosis,” Journal of Immunology, 157:512-521, (1996)). Merocyanine 540-positive cells were identified using 488-nm excitation from an argon laser and a 575-nm DF26 bandpass filter for collection of events with increased red fluorescence. Data were collected and analyzed using CellQuest software (Becton Dickinson). Figures show representative results of at least two independently performed experiments.

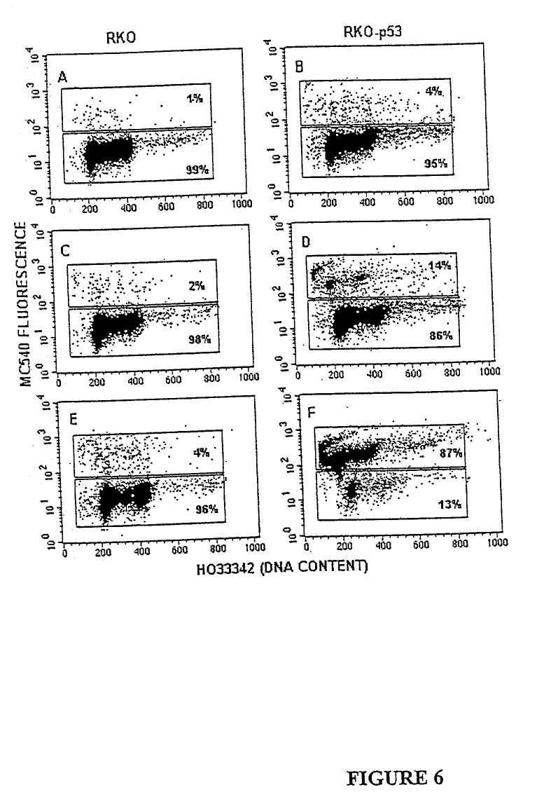

RKO cells without or with a stably-transfected dominant negative mutant p53 gene were subjected to multi-parameter flow cytometry after 24 h of exposure to cerulenin. Ungated two-dimensional analysis of DNA content versus MC540 fluorescence is displayed in FIG. 6 after no drug (A and B), cerulenin (5 μg/ml) (C and D), and cerulenin (10 μg/ml) (E and F). Apoptotic and non-apoptotic cells are in upper and lower boxes, respectively.

Loss of p53 function sensitized RKO and MCF7 cells to the cytotoxic effect of FAS inhibition. There was a large, dose-dependent increase in apoptosis after cerulenin exposure in RKO-p53 cells compared to the parent RKO line (FIG. 6). The cell cycle distribution of the non-apoptotic (lower boxes) and apoptotic (upper boxes) sub-populations of RKO cells after 24 h of exposure to 5 or 10 μg/ml cerulenin was determined by multi-parameter flow cytometry. Cell cycle position (DNA content) was determined with H033342 dye, and apoptosis was detected by bright staining with merocyanine 540 (MC540), which detects conformational changes in the plasma membrane that occur early during apoptosis (Reid et al., 1996; Mower et al., 1994; Castedo et al., 1996). The validity of MC540 staining as a measure of entry into apoptosis has been confirmed in our experimental system by evaluation of morphology, change in light scatter parameters and “TUNEL” DNA end-labeling in parallel experiments [(Pizer et al., 2000; Pizer et al., 1998b) and data not shown]. Entry into apoptosis after FAS inhibition by cerulenin occurred from G1, S and G2/M without increased sensitivity in any subpopulation. Apoptosis with lack of cell cycle phase specificity was typical of many experiments with several cell lines (not shown).

### Example 7

### Loss of p53 Function Sensitizes Colon and Breast Carcinoma Cells to FAS Inhibitor Cytotoxicity

A similar apoptotic response was seen with 3 independent RKO-p53 clones and with MCF7-E6, and was seen after exposure to C-75 (FIGS. 7C and E and data not shown). Apoptotic fraction of colon and breast carcinoma cells after 24 h exposure to FAS inhibitors, analyzed as in Example 6 (FIGS. 6C and E).

The cytotoxic effects of the FAS inhibitors on these paired lines were also tested by clonogenic assay, as well as SW480, a colon carcinoma line with a naturally-occurring p53 mutation, and SKBr3 is a breast carcinoma line with a naturally-occurring p53 mutation (see FIGS. 7D and F). Parallel determinations of sensitivity to FAS inhibitors were performed by clonogenic assay after a 6-h drug exposure. Subconfluent cells were exposed to the indicated drug concentrations for 6 h, then were detached from plastic with trypsin, counted and replated for colony formation. Clones were fixed, stained with crystal violet [0.1%] (Sigma) and counted one week later. Data are presented as mean values with bars showing the standard error. Calculations and graphing were performed in Prism 2.0 (GraphPad).

Comparison of the two cytotoxicity assays shows that inhibition of FAS causes a reduction in the number of clonable RKO and MCF7 cells that is not detected by the apoptosis assay. The clonogenic assay probably detects subpopulations undergoing growth arrest and potentially other growth inhibitory processes in addition to those undergoing rapid apoptosis. However, it appears that the early apoptosis associated with loss of p53 function illustrated in Example 6 further reduces the clonable fraction, resulting in sensitivity to FAS inhibitors that is comparable to that seen with other lines bearing naturally-occurring p53 mutations (SW480 colon carcinoma and SKBr3 breast carcinoma cells).

### Example 8

### FAS Inhibitor Induced Growth Arrest is Independent of Malonyl-CoA Accumulation

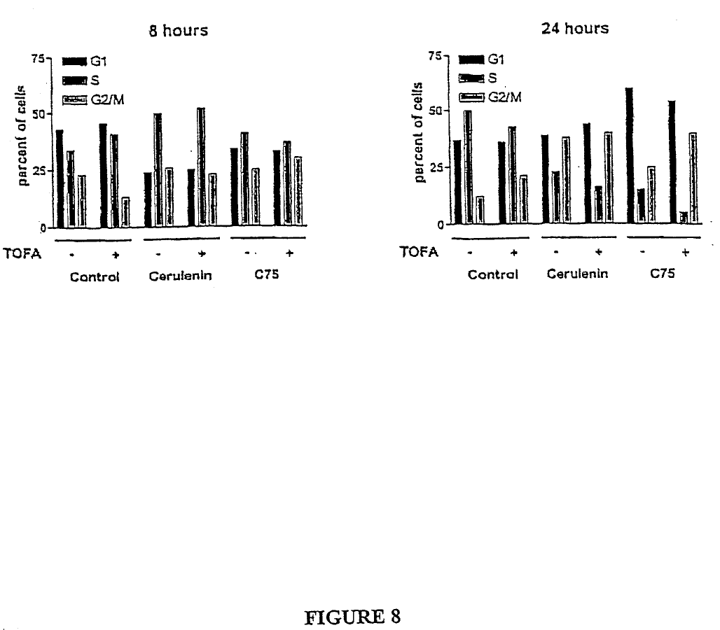

In order to determine the role of malonyl-CoA accumulation in delaying cell cycle progression, RKO cells were analyzed by flow cytometry after 8 or 24 hours of FAS inhibitor exposure, without or with pretreatment for 1 hour with the acetyl-CoA carboxylase (ACC) inhibitor, 5-(tetradecyloxy)-2-furoic acid (TOFA), which blocks the carboxylation of acetyl-CoA to form malonyl-CoA (FIG. 8). RKO cells were exposed to [cerulenin,10 μg/ml] or [C-75,10 μg/ml] for the indicated times, without or with 1 hour [TOFA, 5 μg/ml] pretreatment to inhibit malonyl-CoA synthesis. DNA content was determined as described in Example 1. Determination of the percentages of cells in G1, S and G2/M was done with Multicycle software.

Cells pretreated with TOFA, followed by cerulenin or C-75, showed similar or greater cell cycle delays to cells exposed only to the FAS inhibitors. Of note, however, TOFA pretreatment did rescue FAS inhibitor mediated apoptosis in RKO-p53 cells, similar to earlier results (Pizer et al., 2000), indicating that the effects of FAS inhibitors on cell cycle progression are distinct from those mediating apoptotic cell death.

### Example 9

### Comparison of FAS Enzyme Levels

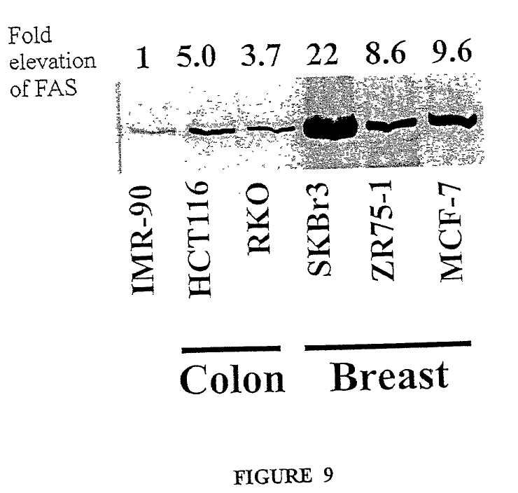

The level of FAS enzyme was measured in non-transformed human cell line, IMR-90, and a panel of tumor lines. FAS enzyme levels in immortalized, non-transformed control cells, IMR-90 (fetal lung), and for tumor lines; HCT116, RKO (colon), SKBr3, ZR75-1 and MCF-7 (breast) were quantitated by immunoblot. The levels of enzyme were adjusted to total cellular protein, and the values obtained were normalized to IMR-90. As shown in FIG. 9, the breast cancer cell lines tested in this comparison have at least eight-fold more FAS than IMR-90, while the colon cancer lines showed 3-5-fold greater FAS.

Although the foregoing invention has been described in some detail by way of illustration and example for purposes of clarity of understanding, it will be obvious that certain changes and modifications may be practiced within the scope of the appended claims. Modifications of the above-described modes for carrying out the invention that are obvious to persons of skill in medicine, immunology, hybridoma technology, pharmacology, and/or related fields are intended to be within the scope of the following claims.

All publications and patent applications mentioned in this specification are indicative of the level of skill of those skilled in the art to which this invention pertains. All such publications and patent applications are herein incorporated by reference to the same extent as if each individual publication or patent application was specifically and individually indicated to be incorporated by reference.

1. A method for treating an individual having a tumor, said method comprising administering to the individual an inhibitor of fatty acid synthase (FAS) in an amount sufficient to retard growth of cells in the tumor.

2. The method of claim 1, wherein the individual has a tumor comprising cells which do not overexpress FAS.

3. The method of claim 1, wherein the individual has a tumor comprising cells which are resistant to induction of apoptosis by inhibitors of FAS.

4. The method of claims 1-3 wherein the tumor is malignant.

5. The method of claim 1, wherein the inhibitor of FAS is administered in an amount sufficient to induce a cellular response equivalent to a genotoxic stress response in the absence of substantial DNA damage.

6. The method of claim 1, wherein cells in the tumor express FAS at a level equal to or less than four-fold higher than IMR-90 cells.

7. Use of a FAS inhibitor in the preparation of a medicament for treating a tumor in an individual whose tumor exhibits reduced p53 function.
